(19) United States
(12) Patent Application Publication
(10) Pub. No.: US 2022/0152125 A1
(43) Pub. Date: May 19, 2022

(54) SURGICAL RECOVERY SUPPLEMENT

(71) Applicant: REVL GROUP LLC, Mesa, AZ (US)

(72) Inventor: Van S. Leavitt, Mesa, AZ (US)

(73) Assignee: REVL GROUP LLC, Mesa, AZ (US)

(21) Appl. No.: 17/529,117

(22) Filed: Nov. 17, 2021

Related U.S. Application Data
(60) Provisional application No. 63/114,971, filed on Nov. 17, 2020.

(51) Int. Cl. A61K 35/741; A23L 33/135; A23L 33/28

(52) U.S. Cl. CPC A61K 35/741; A23L 33/28; A23L 33/135

(57) ABSTRACT

A surgical recovery supplement is disclosed herein. One surgical recovery supplement includes one or more probiotics, one or more forms of fiber, and one or more collagens. Some surgical recovery supplements further include one or more minerals, one or more vitamins, one or more enzymes, one or more antioxidants, and/or one or more extracts. Methods for using the surgical recovery supplements are also disclosed herein. One method includes taking a three times dose of a surgical supplement once a day for 30 days. Another method includes taking a single dose of a surgical supplement three times a day for 30 days.

### REFERENCE TO RELATED APPLICATION

This application claims priority to and the benefit of U.S. Provisional Patent Application No. 63/114,971, filed on Nov. 17, 2021, the contents of which are incorporated herein by reference in their entirety.

### FIELD

The subject matter disclosed herein relates to nutritional supplements and, more particularly, relates to a surgical recovery supplement.

### BACKGROUND

Everyday hundreds of thousands of people around the world undergo surgical interventions. The techniques of surgery have become less invasive and more precise by the day. This is due, at least in part, to ever improving technology and surgical techniques, as well as, surgeons who willingly engage in lifelong medical learning and professional refinement. Despite the vast efforts by healthcare providers to improve the outcome in the operating room, little has been done in the way of nutrient supplementation during the perioperative period. A patient's nutritional status is the core of a positive surgical recovery and a successful surgical outcome. Without the proper nutrition levels, even the most cutting-edge surgical techniques and advanced technology are unable to guarantee excellent surgical recoveries. After years of performing surgery with little emphasis on ensuring proper post-operative nutrition, it is evident that a post-operative supplement is a necessary aid to an optimal surgical recovery.

### BRIEF SUMMARY

Various embodiments provide a surgical recovery supplement. One surgical recovery supplement includes one or more probiotics, one or more forms of fiber, and one or more collagens. Some surgical recovery supplements further include one or more minerals, one or more vitamins, one or more enzymes, one or more antioxidants, and/or one or more extracts. Methods for using the surgical recovery supplements are also disclosed herein. One method includes taking a three times (3×) dose of a surgical supplement once a day for 30 days. Another method includes taking a single dose of a surgical supplement three times a day for 30 days.

### BRIEF DESCRIPTION OF THE DRAWINGS

So that the advantages of the various embodiments of the present technology will be readily understood, a more particular description of the embodiments briefly described above will be rendered by reference to specific embodiments that are illustrated in the appended drawings. Understanding that these drawings depict only some embodiments and are not therefore to be considered to be limiting of scope, the embodiments will be described and explained with additional specificity and detail through the use of the accompanying drawings.

FIG. 1A is a block diagram of one embodiment of a surgical recovery supplement;

FIG. 1B is a block diagram of another embodiment of a surgical recovery supplement;

FIG. 2 is a flow diagram of one embodiment of a method for using the surgical recovery supplements of FIGS. 1A and 1B; and

FIG. 3 is a flow diagram of another embodiment of a method for using the surgical recovery supplements of FIGS. 1A and 1B.

### DETAILED DESCRIPTION OF THE INVENTION

It should be understood that the language used in the present disclosure has been principally selected for readability and instructional purposes, and not to limit the scope of the subject matter disclosed herein in any manner. Further, reference throughout this specification to “one embodiment,” “an embodiment,” or similar language means that a particular feature, structure, or characteristic described in connection with the embodiment is included in at least one embodiment. Thus, appearances of the phrases “in one embodiment,” “in an embodiment,” and similar language throughout this specification may, but do not necessarily, all refer to the same embodiment, but mean “one or more but not all embodiments” unless expressly specified otherwise. The terms “including,” “comprising,” “having,” and variations thereof mean “including, but not limited to” unless expressly specified otherwise. An enumerated listing of items does not imply that any or all of the items are mutually exclusive and/or mutually inclusive, unless expressly specified otherwise. The terms “a,” “an,” and “the” also refer to “one or more” unless expressly specified otherwise.

In addition, as used herein, the term “set” can mean “one or more,” unless expressly specified otherwise. The term “sets” can mean multiples of or a plurality of “one or mores,” “ones or more,” and/or “ones or mores” consistent with set theory, unless expressly specified otherwise.

Furthermore, the described features, advantages, and characteristics of the embodiments may be combined in any suitable manner. One skilled in the relevant art will recognize that the embodiments may be practiced without one or more of the specific features or advantages of a particular embodiment. In other instances, additional features and advantages may be recognized in certain embodiments that may not be present in all embodiments.

The present technology may include any type of release and/or delivery mechanism and is not limited to the release and/or delivery mechanism(s) depicted in the drawings. Furthermore, the described features, structures, or characteristics of the various embodiments may be combined in any suitable manner. One skilled in the relevant art will recognize, however, that embodiments may be practiced without one or more of the specific details, or with other methods, components, materials, and so forth. In other instances, well-known structures, and/or materials are not shown or described in detail to avoid obscuring aspects of a particular embodiment.

The various embodiments discussed herein provide surgical recovery supplements for post-surgical use. That is, the various surgical recovery supplements can be used after any surgical procedure including, but not limited to, joint replacement surgery, gallbladder removal, hernia repair, cancer surgery, and a trauma surgery, among other surgeries that are possible and contemplated herein.

The surgical recovery supplements, in various embodiments, include compounds that are a combination of elements that may be essential to a healthy recovery from a surgical procedure, which can provide maximum benefit to a patient while the patient is recovery from surgery. The surgical recovery supplements discussed herein provide many elements that can promote wound healing and recovery and are can be a tool to help the patient during the critical first weeks after surgical recovery. Further, the various surgical recovery supplements can be used after any surgical procedure including, but not limited to, joint replacement surgery, gallbladder removal, hernia repair, cancer surgery, and a trauma surgery, among other surgeries that are possible and contemplated herein.

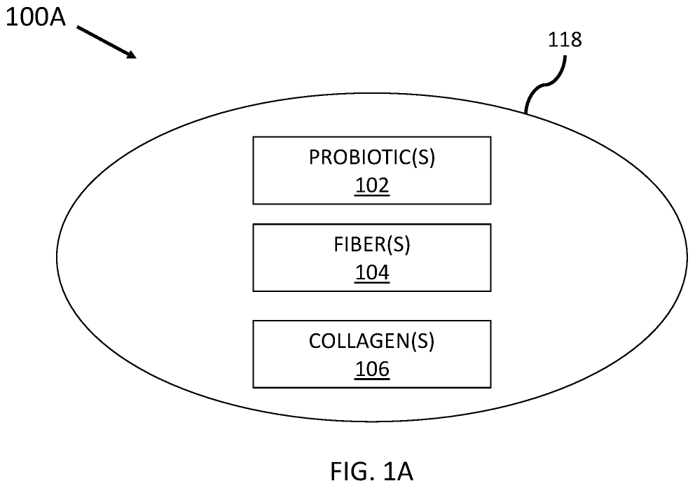

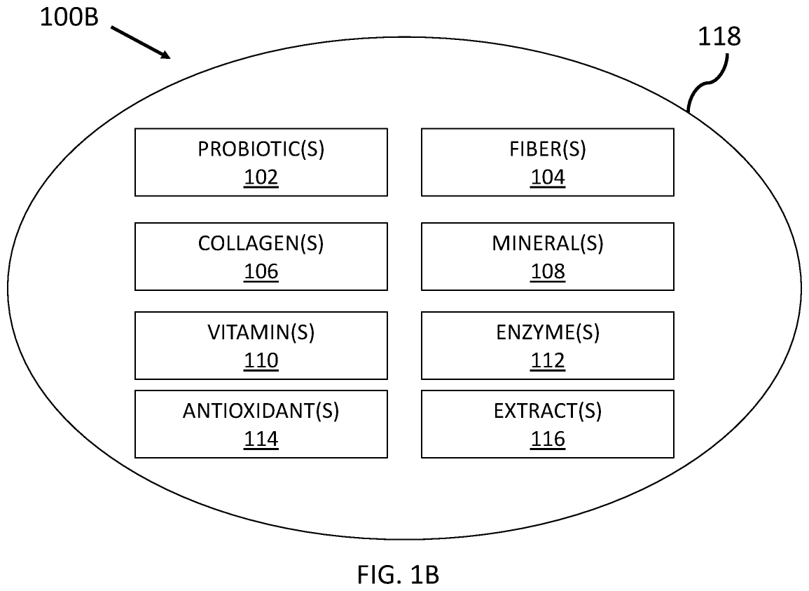

Turning now to the drawings, FIG. 1A is a block diagram illustrating one embodiment of a surgical recovery supplement 100A. The surgical recovery supplement 100A may be in the form of a solid (e.g., a powder, granules, etc.) and/or a liquid. At least in the illustrated embodiment, the surgical recovery supplement 100A forms a compound that includes, among other components, one or more probiotics 102, one or more forms of fiber 104, and one or more forms of collagen 106.

The probiotic(s) 102 are utilized to help restore and maintain the balance of healthy bacteria in the system since the patient is typically given a large dose of preoperative antibiotics and often times post-operative antibiotic regimens. Each of the one or more probiotics 102 may be any probiotic 102 that is known or developed in the future that can help restore and maintain the balance of healthy bacteria in the system (e.g., the patient's digestive system).

In some embodiments, the surgical recovery supplement 100A includes one probiotic 102. In other embodiments, the surgical recovery supplement 100A includes multiple probiotics. Example probiotics 102 for inclusion in the surgical recovery supplement 100A include, but are not limited to, Bifidobacterium longum, Lactobacillus plantarum, Bifidobacterium bifidum, Lactobacillus acidophilus, and Lactobacillus rhamnosus, etc., among other probiotics that are possible and contemplated herein.

The surgical recovery supplement 100A may include any suitable amount of probiotic 102. In various embodiments, the surgical recovery supplement 100A includes an amount of probiotic 102 in the range of about 10 CFU (Colony-Forming Units) to about 150 CFU or about 10 milligrams (mg) to about 500 mg, among other ranges and/or amounts that are possible and contemplated herein. In certain embodiments, the surgical recovery supplement 100A includes about 200 mg of probiotic 102, among other amounts that are possible and contemplated herein.

The fiber 104 is utilized to aid in an earlier return of proper bowel function in a post-surgery patient. Further, the fiber 104 helps to substantially prevent/prevent, minimize, or at least reduce the occurrence of constipation in a post-surgery patient.

The fiber 104 can include any suitable type, form, and/or source of fiber that is known or developed in the future. In certain embodiments, the fiber 104 may include inulin, among other types, forms, and/or sources of fiber that are possible and contemplated herein.

The surgical recovery supplement 100A may include any suitable amount of fiber 104. In various embodiments, the surgical recovery supplement 100A includes an amount of fiber 104 in the range of about 0.5 grams to about 5 grams, among other ranges and/or amounts that are possible and contemplated herein. In certain embodiments, the surgical recovery supplement 100A includes about 2 grams of fiber 104, among other amounts that are possible and contemplated herein.

The collagen 106 is utilized as a building block to help with skin, muscle, and bone growth, as well as digestive maintenance, in a post-surgery patient. The collagen 106 can include any suitable type, form, and/or source of collagen that is known or developed in the future. In certain embodiments, the collagen 106 may include a Type 1 collagen and/or a Type 3 collagen, among other types, forms, and/or sources of collagen that are possible and contemplated herein.

The surgical recovery supplement 100A may include any suitable amount of collagen 106. In various embodiments, the surgical recovery supplement 100A includes an amount of collagen 106 in the range of about 0.5 grams to about 5 grams, among other ranges and/or amounts that are possible and contemplated herein. In certain embodiments, the surgical recovery supplement 100A includes about 2 grams of collagen 106, among other amounts that are possible and contemplated herein.

With reference now to FIG. 1B, FIG. 1B is a block diagram illustrating another embodiment of a surgical recovery supplement 100B. The surgical recovery supplement 100B forms a compound that includes one or more probiotics 102, one or more forms of fiber 104, and one or more forms of collagen 106 similar to the surgical recovery supplement 100A discussed with reference to FIG. 1A. At least in the illustrated embodiment, the compound of the surgical recovery supplement 100B further includes, among other components, one or more minerals 108, one or more vitamins 110, one or more enzymes 112, one or more antioxidants 114, and/or one or more extracts 116, and the various possible combinations of the mineral(s) 108, vitamin(s) 110, enzyme(s) 112, antioxidant(s) 114, and/or extract(s) 116.

The mineral(s) 108 may include any suitable mineral(s) 108 that is/are known or developed in the future. Further, the mineral(s) 108 may include any suitable quantity of minerals 108. In certain embodiments, the surgical recovery supplement 100B includes at least zinc (e.g., zinc sulfate, etc.), among other minerals capable of improving a post-surgery patient's immune system function and/or the wound healing process that are possible and contemplated herein.

The surgical recovery supplement 100B may include any suitable amount of the mineral(s) 108 (e.g., zinc, etc.). In certain embodiments, the surgical recovery supplement 100B includes at least the recommended daily amount (RDA) of zinc and/or other mineral(s) established by the United States Department of Agriculture (USGA). In other embodiments, the surgical recovery supplement 100B includes less than the RDA of zinc and/or other mineral(s) established by the USGA.

In various embodiments, the surgical recovery supplement 100B includes an amount of zinc and/or other mineral(s) in the range of about 5 mg to about 500 mg, among other ranges and/or amounts that are possible and contemplated herein. In certain embodiments, the surgical recovery supplement 100B includes about 50 mg of zinc and/or other mineral(s), among other amounts that are possible and contemplated herein.

The vitamin(s) 110 may include any suitable vitamin(s) 110 that is/are known or developed in the future. Further, the vitamin(s) 110 may include any suitable quantity of vitamins 110. In certain embodiments, the surgical recovery supplement 100B includes at least Vitamin A (e.g., retinoid, etc.) and/or Vitamin C (e.g., ascorbic acid, etc.), among other vitamins that are possible and contemplated herein.

Vitamin A is a fat-soluble vitamin utilized to help support cell growth, immune function, and/or maintenance of skin surfaces. The surgical recovery supplement 100B may include any suitable amount of the vitamin(s) 110 (e.g., Vitamin A and/or Vitamin C, etc.). In certain embodiments, the surgical recovery supplement 100B includes at least the RDA of Vitamin A and/or Vitamin C, etc. established by the USGA. In other embodiments, the surgical recovery supplement 100B includes less than the RDA of Vitamin A and/or Vitamin C, etc. established by the USGA.

In various embodiments, the surgical recovery supplement 100B includes an amount of Vitamin A in the range of about 100 mcg to about 5000 mcg, among other ranges and/or amounts that are possible and contemplated herein. In certain embodiments, the surgical recovery supplement 100B includes about 1500 mcg (5000 International Units (IU)) of Vitamin A, among other amounts that are possible and contemplated herein.

In additional or alternative embodiments, the surgical recovery supplement 100B includes an amount of Vitamin C in the range of about 50 mg to about 5000 mg, among other ranges and/or amounts that are possible and contemplated herein. In certain embodiments, the surgical recovery supplement 100B includes about 500 mg of Vitamin C, among other amounts that are possible and contemplated herein.

Vitamin C is utilized to help in the development, growth, and repair of many types of tissues. Additionally, Vitamin C is involved in the creation of collagen in the human body.

The enzyme(s) 112 may include any suitable enzyme(s) 112 that is/are known or developed in the future. Further, the enzyme(s) 112 may include any suitable quantity of enzymes 112. In certain embodiments, the surgical recovery supplement 100B includes at least Bromelain (e.g., pineapple, a mixture that include pineapple, etc), among other enzymes capable of reducing inflammation, swelling, and/or bruising and/or helping with overall wound healing and reducing tenderness after injury/surgery that are possible and contemplated herein.

The surgical recovery supplement 100B may include any suitable amount of the enzyme(s) 112 (e.g., pineapple, etc.). In various embodiments, the surgical recovery supplement 100B includes an amount of Bromelain and/or other enzyme(s) in the range of about 25 mg to about 500 mg, among other ranges and/or amounts that are possible and contemplated herein. In certain embodiments, the surgical recovery supplement 100B includes about 200 mg of Bromelain (e.g., 2400 GDU/gram of pineapple) and/or other enzyme(s), among other amounts that are possible and contemplated herein.

The antioxidant(s) 114 may include any suitable antioxidant(s) 114 that is/are known or developed in the future. Further, the antioxidant(s) 114 may include any suitable quantity of antioxidants 114. In certain embodiments, the surgical recovery supplement 100B includes at least Rutin (e.g., a plant pigment (flavonoid) that is found in some fruits and vegetables), among other antioxidants capable of helping a post-surgery patient's body use Vitamin C and produce collagen, aid in blood circulation, and/or reducing the risk of blood clot formation that are possible and contemplated herein.

A source of Rutin for use in a supplement include, but are not limited to, buckwheat, Japanese pagoda tree, and Eucalyptus. Other sources of Rutin can include lime tree flowers, elder flowers, hawthorn, rue, St. John's Wort, Ginkgo, apples, and other fruits and vegetables.

The surgical recovery supplement 100B may include any suitable amount of the antioxidant(s) 114 (e.g., Rutin, etc.). In various embodiments, the surgical recovery supplement 100B includes an amount of Rutin and/or other antioxidant(s) in the range of about 25 mg to about 1000 mg, among other ranges and/or amounts that are possible and contemplated herein. In certain embodiments, the surgical recovery supplement 100B includes about 250 mg of Rutin and/or other antioxidant(s), among other amounts that are possible and contemplated herein.

The extract(s) 116 may include any suitable extract(s) 116 that is/are known or developed in the future. Further, the extract(s) 116 may include any suitable quantity of extract 116. In certain embodiments, the surgical recovery supplement 100B includes at least grape seed extract, among other enzymes capable of accelerating the rate of wound healing, improving bone strength, acting as a blood thinner, and/or helping reduce the risk of blood clot formation that are possible and contemplated herein.

The surgical recovery supplement 100B may include any suitable amount of the extract(s) 116 (e.g., grape seed extract, etc.). In various embodiments, the surgical recovery supplement 100B includes an amount of grape seed extract and/or other extract(s) in the range of about 5 mg to about 500 mg, among other ranges and/or amounts that are possible and contemplated herein. In certain embodiments, the surgical recovery supplement 100B includes about 60 mg of grape seed extract (e.g., 16:1 Extract) and/or other extract(s), among other amounts that are possible and contemplated herein.

As shown in FIGS. 1A and 1B, the surgical recovery supplements 100A and 100B can include a delivery mechanism 118. In various embodiments, the delivery mechanism 118 can include a tablet, a coated tablet, a capsule, a gel capsule, a powder, or granules, etc., among other solid delivery mechanisms that are possible and contemplated herein. In alternative embodiments, the delivery mechanism 102 can include a syrup, one or more drops, and/or a solution, among other liquid delivery mechanisms that are possible and contemplated herein. In additional embodiments, the delivery mechanism 118 can include a combination of one or more solids and/or one or more liquids, among other possible combinations.

In the various embodiments, the delivery mechanism 118 allows a patient to orally consume the surgical recovery supplements 100A and 100B. That is, a patient can swallow the surgical recovery supplements 100A and 100B whole and/or drink the surgical recovery supplements 100A and 100B.

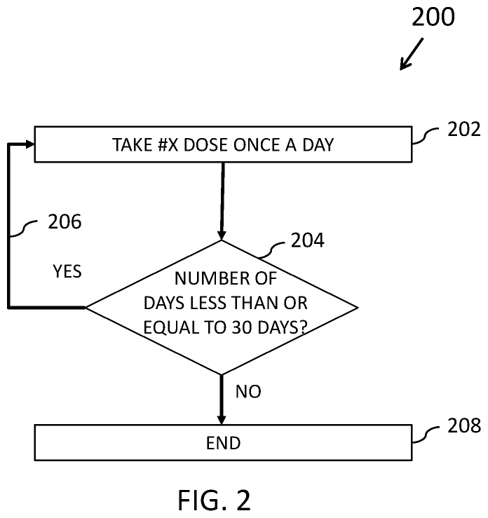

Referring to FIG. 2, FIG. 2 is a flow diagram of one embodiment of a method 200 for using the surgical recovery supplements 100A and 100B. At least in the illustrated embodiment, the method 200 begins by a patient taking a three times (3×) dose of the surgical recovery supplement 100A or 100B (block 202).

The method 200 then includes determining whether the current day is a day that is less than or equal 30 days from when the patient had surgery (block 204). If the current day is a day that is less than or equal to 30 days from when the patient had surgery (e.g., a “YES” in block 204), the method 200 includes the patient taking another 3× dose of the surgical recovery supplement 100A or 100B (retun 206). If the current day is a day that is not less than or equal to 30 days (e.g., 31 days) from when the patient had surgery (e.g., a “NO” in block 204), the method 200 ends (block 208).

The 3× dose in the method 200 can be taken at any suitable time of day. In various embodiments, the 3× dose can be taken in the morning before, at noon, or at a time after noon. In some embodiments, the 3× dose can be taken before or after a meal. In other embodiments, the 3× dose can be taken shortly before bedtime or shortly after waking up.

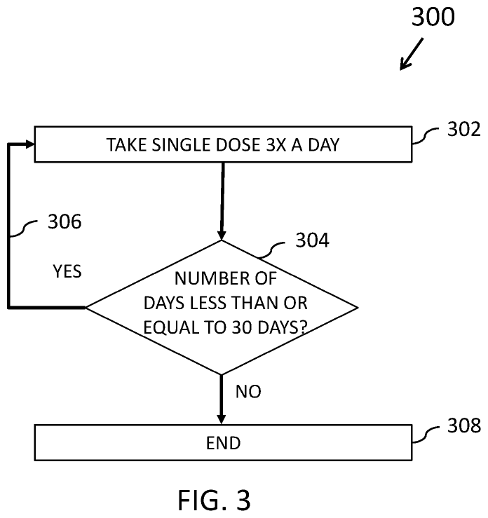

With reference to FIG. 3, FIG. 3 is a flow diagram of another embodiment of a method 300 for using the surgical recovery supplements 100A and 100B. At least in the illustrated embodiment, the method 300 begins by a patient taking a single dose of the surgical recovery supplement 100A or 100B three times (3×) a day (block 302).

The method 300 then includes determining whether the current day is a day that is less than or equal 30 days from when the patient had surgery (block 304). If the current day is a day that is less than or equal to 30 days from when the patient had surgery (e.g., a “YES” in block 304), the method 300 includes the patient taking another dose of the surgical recovery supplement 100A or 100B three times in a single day (retun 306). If the current day is a day that is not less than or equal to 30 days (e.g., 31 days) from when the patient had surgery (e.g., a “NO” in block 304), the method 300 ends (block 308).

In some embodiments of the method 300, the three doses can be spaced apart a predetermined amount of time (e.g., 8 hours apart, at least 4 hours apart, etc., among other time spacing that allows three does to be taken that are possible and contemplated herein). In additional or alternative embodiments of the method 300, each of the three doses can be taken before or after each of three meals in a day. In other embodiments, a first dose can be taken shortly after waking up, a second dose is taken at or around noon time, and a third dose is taken shortly before bedtime.

The embodiments may be practiced in other specific forms. The described embodiments are to be considered in all respects only as illustrative and not restrictive. The scope of the technology is, therefore, indicated by the appended claim rather than by the foregoing description. All changes which come within the meaning and range of equivalency of the claims are to be embraced within their scope.

What is claimed is:

1. A surgical supplement, comprising:
one or more probiotics;
one or more forms of fiber; and
one or more collagens.

2. The surgical supplement of claim 1, wherein the one or more probiotics comprises one or more of Bifidobacterium longum, Lactobacillus plantarum, Bifidobacterium bifidum, Lactobacillus acidophilus, and Lactobacillus rhamnosus.

3. The surgical supplement of claim 2, wherein the one or more probiotics comprises an amount in the range of 10 milligrams to about 500 milligrams.

4. The surgical supplement of claim 1, wherein the one or more probiotics comprises an amount in the range of 10 milligrams to about 500 milligrams.

5. The surgical supplement of claim 1, wherein the one or more forms of fiber comprises at least inulin.

6. The surgical supplement of claim 5, wherein the one or more forms of fiber comprises an amount in the range of 0.5 grams to 5 grams.

7. The surgical supplement of claim 1, wherein the one or more forms of fiber comprises an amount in the range of 0.5 grams to 5 grams.

8. The surgical supplement of claim 1, wherein the one or more collagens comprises a Type 1 collagen and/or a Type 3 collagen.

9. The surgical supplement of claim 8, wherein the one or more collagens comprises an amount in the range of 0.5 grams to 5 grams.

10. The surgical supplement of claim 1, wherein the one or more collagens comprises an amount in the range of 0.5 grams to 5 grams.

11. The surgical supplement of claim 1, wherein:
the one or more probiotics comprises one or more of Bifidobacterium longum, Lactobacillus plantarum, Bifidobacterium bifidum, Lactobacillus acidophilus, and Lactobacillus rhamnosus;
the one or more forms of fiber comprises at least inulin; and
the one or more collagens comprises a Type 1 collagen and/or a Type 3 collagen.

12. The surgical supplement of claim 11, wherein:
the one or more probiotics comprises an amount in the range of 10 milligrams to about 500 milligrams;
the one or more forms of fiber comprises an amount in the range of 0.5 grams to 5 grams; and
the one or more collagens comprises an amount in the range of 0.5 grams to 5 grams.

13. The surgical supplement of claim 1, further comprising:
one or more minerals;
one or more vitamins;
one or more enzymes;
one or more antioxidants; and/or
one or more extracts.

14. The surgical supplement of claim 1, wherein the one or more probiotics, the one or more forms of fiber, and the one or more collagens are in a solid form.

15. The surgical supplement of claim 1, wherein the one or more probiotics, the one or more forms of fiber, and the one or more collagens are in a liquid form.

16. The surgical supplement of claim 1, wherein the one or more probiotics, the one or more forms of fiber, and the one or more collagens are in a form of a combination of a solid and a liquid.

17. A method, comprising:
taking a surgical supplement one time a day for 30 days,
wherein the surgical supplement comprises one or more probiotics, one or more forms of fiber, and one or more collagens.

18. The method of claim 17, wherein the surgical supplement is a 3 times (3×) dose of the one or more probiotics, one or more forms of fiber, and one or more collagens.

19. A method, comprising:
taking a surgical supplement three times a day for 30 days,
wherein the surgical supplement comprises one or more probiotics, one or more forms of fiber, and one or more collagens.

20. The method of claim 19, wherein the surgical supplement is a single dose of the one or more probiotics, one or more forms of fiber, and one or more collagens.
